SEQUENCE LISTING (28 sequences)

SEQ ID NO: 1 - DNA, 2282 bp, Artificial
  Fusion of human c-myc gene fused to mouse estrogen receptor
gacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacgggatccacgaaatgaaatgggtgcttcaggagacatgagggctgccaacctttggccaagccctcttgtgattaagcacactaagaagaatagccctgccttgtccttgacagctgaccagatggtcagtgccttgttggatgctgaaccgcccatgatctattctgaatatgatccttctagacccttcagtgaagcctcaatgatgggcttattgaccaacctagcagatagggagctggttcatatgatcaactgggcaaagagagtgccaggctttggggacttgaatctccatgatcaggtccaccttctcgagtgtgcctggctggagattctgatgattggtctcgtctggcgctccatggaacacccggggaagctcctgtttgctcctaacttgctcctggacaggaatcaaggtaaatgtgtggaaggcatggtggagatctttgacatgttgcttgctacgtcaagtcggttccgcatgatgaacctgcagggtgaagagtttgtgtgcctcaaatccatcattttgcttaattccggagtgtacacgtttctgtccagcaccttgaagtctctggaagagaaggaccacatccaccgtgtcctggacaagatcacagacactttgatccacctgatggccaaagctggcctgactctgcagcagcagcatcgccgcctagctcagctccttctcattctttcccatatccggcatatgagtaacaaacgcatggagcatctctacaacatgaaatgcaagaacgtggtacccctctatgacctgctcctggagatgttggatgcccaccgccttcatgccccagccagtcgcatgggagtgcccccagaggagcccagccagacccagctggccaccaccagctccacttcagcacattccttacaaacctactacatacccccggaagcagagggcttccccaacacgatctgagagctcc

SEQ ID NO: 2 - protein, 754 aa, Artificial
  Fusion of human c-myc gene fused to mouse\ estrogen receptor
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRDPRNEMGASGDMRAANLWPSPLIKHTKKNSPALSLTADQMVSALLDAEPPMIYSEYDPSRPFSEASMMGLLTNLADRELVHMINWAKRVPGFGDLNLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHRRLAQLLLILSHIRHMSNKRMEHLYNMKCKNVVPYDLLLEMLDAHRLHAPASRMGVPPEEPSQTQLATTSSTSAHSLQTYYIPPEAEGFPNTI

SEQ ID NO: 3 - DNA, 20 bp, Artificial
  Oligonucleotide Primer
ctcctggcaaaaggtcagag

SEQ ID NO: 4 - DNA, 20 bp, Artificial
  Oligonucleotide Primer
aaggacaaggcagggctatt

SEQ ID NO: 5 - DNA, 20 bp, Artificial
  Oligonucleotide Primer
aaaggcccccaaggtagtta

SEQ ID NO: 6 - DNA, 20 bp, Artificial
  Oligonucleotide Primer
aaggacaaggcagggctatt

SEQ ID NO: 7 - DNA, 20 bp, Artificial
  Oligonucleotide Primer
cctgtggatgcgcctcctgc

SEQ ID NO: 8 - DNA, 27 bp, Artificial
  Oligonucleotide Primer
gctggttcaagggctttattccatctc

SEQ ID NO: 9 - DNA, 18 bp, Artificial
  Oligonucleotide Primer
agaggagcccagccagac

SEQ ID NO: 10 - DNA, 22 bp, Artificial
  Oligonucleotide Primer
tgtaaggaatgtgctgaagtgg

SEQ ID NO: 11 - DNA, 20 bp, Artificial
  Oligonucleotide Primer
cgttggggatgacagagagt

SEQ ID NO: 12 - DNA, 20 bp, Artificial
  Oligonucleotide Primer
cgagtcctgcttcttcttgg

SEQ ID NO: 13 - DNA, 20 bp, Artificial
  Oligonucleotide Primer
aaggacaagaagcgaagcat

SEQ ID NO: 14 - DNA, 20 bp, Artificial
  Oligonucleotide Primer
ttcctgtcatcccctggata

SEQ ID NO: 15 - DNA, 18 bp, Artificial
  Oligonucleotide Primer
aagctcacgcgtggaaag

SEQ ID NO: 16 - DNA, 22 bp, Artificial
  Oligonucleotide Primer
gccgtgagatgtacttgttgaa

SEQ ID NO: 17 - DNA, 22 bp, Artificial
  Oligonucleotide Primer
aggcttcttctacacacccaag

SEQ ID NO: 18 - DNA, 18 bp, Artificial
  Oligonucleotide Primer
cacaatgccacgcttctg

SEQ ID NO: 19 - DNA, 20 bp, Artificial
  Oligonucleotide Primer
agaagaggccatcaagcaga

SEQ ID NO: 20 - DNA, 20 bp, Artificial
  Oligonucleotide Primer
caggtgttggttcacaaagg

SEQ ID NO: 21 - DNA, 19 bp, Artificial
  Oligonucleotide Primer
cgagggccttcagtactcc

SEQ ID NO: 22 - DNA, 19 bp, Artificial
  Oligonucleotide Primer
ggggacttggagcttgagt

SEQ ID NO: 23 - DNA, 19 bp, Artificial
  Oligonucleotide Primer
gtacaaggcagctggcaac

SEQ ID NO: 24 - DNA, 21 bp, Artificial
  Oligonucleotide Primer
tgggaagctgagaatgatctg

SEQ ID NO: 25 - DNA, 24 bp, Artificial
  Oligonucleotide Primer
tctgttctacagcacactaccaga

SEQ ID NO: 26 - DNA, 20 bp, Artificial
  Oligonucleotide Primer
agctgccttgtaccagcatt

SEQ ID NO: 27 - DNA, 18 bp, Artificial
  Oligonucleotide Primer
ggggtcactcaggacagc

SEQ ID NO: 28 - DNA, 21 bp, Artificial
  Oligonucleotide Primer
tcttgaagtctgagggcagtg

(19) United States
(12) Patent Application Publication
(10) Pub. No.: US 2009/0238799 A1
(43) Pub. Date: Sep. 24, 2009

(54) Conditionally-Immortalised Pancreatic Cells

(76) Inventors: John Sinden, Surrey (GB); Lara Stevanato, Surrey (GB); Erik Miljan, Surrey (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/092,356

(22) PCT Filed: Nov. 3, 2006
(86) PCT No.: PCT/GB06/04103

§ 371 Date: May 1, 2008

(30) Foreign Application Priority Data
Nov. 4, 2005 (GB) 0522564.4

(51) Int. Cl. A61K 35/12; C12N 5/06; C12N 5/08

(52) U.S. Cl. 424/93.7; 435/325; 435/404; 435/366; 435/375; 435/29

(57) ABSTRACT

Conditionally immortalized pancreatic cells are produced which remain immortal in the presence of 4-hydroxytamoxifen (4-OHT) but express normal pancreatic cell markers when 4-OHT is not present. The cells therefore permit cell lines to be constructed to produce cells useful for transplantation or in screening assays.

### FIELD OF THE INVENTION

This invention relates to conditionally-immortalized pancreatic cells that can be scaled up for clinical and commercial application.

### BACKGROUND TO THE INVENTION

Diabetes in all its forms currently afflicts at least 200 million people in the world and this number is expected to double by the year 2025. Type 1 (insulin-dependent) diabetes is a chronic disease affecting genetically predisposed individuals, usually at a young age, in which insulin-secreting β-cells within pancreatic islets of Langerhans are selectively and irreversibly destroyed by autoimmune assault. For over 80 years the main therapeutic approach to insulin-dependent diabetes has been confined to treating “the” symptoms by insulin replacement. Recent studies have emphasized the importance of strict glycemic control in order to reduce opthalmologic, neurological, and renal complications of the disease (1).

Significant advances in the transplantation of human primary islets of Langerhans into individuals with Type 1 diabetes, has largely removed this insulin dependency (2). However, the application of this treatment is restricted by the very limited availability of primary human islets from heart beating donors, and what is now required is an essentially limitless supply of a physiologically competent substitute for primary human islets to make a major clinical impact. The beta-cell-specific nature of Type-1 diabetes makes it particularly amenable to treatment by cell-replacement therapy. The ability to differentiate pluripotent stem cells into functional beta-cells can offer new therapeutic possibilities for the treatment of Type-1 diabetes. Human fetal islet cells or their precursor cells are a potential alternative source of insulin-producing tissue for clinical transplantation (3). The success of transplanting fetal tissue is dependent on the ability of the fetal cells not only to grow, but also to mature at the implantation site.

Immortalized pancreatic fetal cells could provide a limitless supply of a physiologically competent substitute for primary human islets of Langerhans. The development of the islets of Langerhans in the mammalian pancreas has been intensively studied as an example of coordinated tissue morphogenesis and because it is hoped that an understanding of this process will facilitate the development of a cell transplantation therapy for diabetes (4, 5). The islets that comprise the endocrine compartment of the pancreas contain four cell types, each producing a distinct hormone alpha, beta-, and pancreatic polypeptide (PP) cells secrete glucagon, somatostatin, and PP, respectively. Insulin production is limited to beta-cells, which comprise the majority of the adult islet cells and are key regulators of glucose homeostasis. Isolation of endocrine cell precursors from the human fetal pancreas will be important to the study of islet cyto-differentiation and eventually for islet transplantation in insulin-dependent diabetes. These precursor cells, from which all four islet endocrine cell types arise, are present within fetal pancreatic ductal epithelium. The early pancreatic bud shows uniform expression of the homeobox gene IPF-1 (also known as IDX-1, STF-1 or PDX).

### SUMMARY OF THE INVENTION

The present invention is based upon the construction of a conditionally immortalised pancreas cell that is immortal when 4-hydroxytamoxifen (4-OHT) is present in the cell culture but which expresses normal pancreatic cell markers when 4-OHT is not present. A c-myc/estrogen receptor fusion is responsible for conferring the conditionally immortal character to the pancreatic cells.

According to a first aspect of the invention, a mammalian pancreatic cell comprises a fusion protein comprising an oncoprotein of the myc family and an oestrogen receptor, or functional fragments thereof, expressed as a single polypeptide chain.

According to a second aspect of the invention, a method of conditionally immortalising a pancreatic cell comprises the steps of:

(i) expressing in the pancreatic cell a fusion protein comprising an oncoprotein of the myc family and an oestrogen receptor, or functional fragments thereof, expressed as a single polypeptide chain; and

(ii) contacting the pancreatic cell formed by step (i) with a ligand of the estrogen receptor, thereby conditionally immortalising the pancreatic cell.

The pancreatic cell according to the invention is particularly useful for the treatment of Type-1 diabetes, severe forms of Type-2 diabetes and in vitro testing of potential drugs.

According to a third aspect of the invention, a method of evaluating the suitability of a compound for use as a drug, in vitro, comprises the steps of:

(i) contacting a pancreatic cell comprising a fusion protein comprising an oncoprotein of the myc family and an oestrogen receptor, or functional fragments thereof, expressed as a single polypeptide chain, with the potential drug compound;

(ii) measuring the response of the pancreatic cell, to thereby determine the effect of the compound on pancreatic cells and thereby evaluate the suitability of the compound for use as a drug. Preferably, the pancreatic cell will be growth-arrested by the removal of the ligand, 4-OHT, from the culture medium, and the pancreatic cells will develop a fully differentiated phenotype.

Further aspects of the invention are the use of pancreatic cells of the invention as a medicament, and in the manufacture of a medicament for the treatment of diabetes.

### DESCRIPTION OF THE DRAWINGS

The invention is described with reference to the accompanying drawings, wherein:

FIG. 1 is a graphic illustration showing the number of cells produced using uninfected GS080 (15 week) tissue compared to the same tissue transduced with C-myCER^{TAM}, with cell counting carried out automatically using CyQuant (A) or by manual counting (B);

FIG. 2 shows pancreas cell lines differentiated into mature pancreas cells expressing appropriate markers; (A) shows cell aggregates of approximately 200 μm in diameter; (B) shows that the cells stain positive for the marker pancreas c-peptide insulin, (C) shows that the cells stain positive for the marker PDX-1;

FIG. 3 is a graph showing relative expression of c-mycERTAM in pancreas cells; and

FIG. 4 shows the telomerase induction by 4-OHT on the pancreatic cells.

### DETAILED DESCRIPTION OF THE INVENTION

The present invention identifies that expression of a myc/oestrogen-receptor fusion protein in a pancreas primary culture conditionally immortalises them, providing pancreatic cell lines. When the pancreatic cells are cultured in the presence of a ligand of the estrogen receptor, they are immortal. The removal of the ligand from the pancreatic cells removes the immortality of the cells, which then displays “the normal” characteristics and markers of pancreas cells, as would be expected from pancreatic cells in situ in a pancreas.

As used herein, the terms “pancreas cells” or “pancreatic cells” refers to any cells obtainable from pancreas that is capable of performing one or more functions carried out by the pancreas. Pancreas cells from any species are within the scope of the invention, although it is preferred that the pancreatic cells are mammalian, most preferably human. Fetal or adult pancreas may be used, as can pancreatic cells that have differentiated from a precursor cell in vitro, e.g. pancreatic cells differentiated from embryonic stem cells or pluripotent stem cells.

Pancreatic cells according to the present invention maintain markers characteristic of normal pancreas. The islets that comprise the endocrine compartment of the pancreas contain four cell types, each producing a distinct hormone. alpha-, beta-, and pancreatic polypeptide (PP) cells secrete glucagon, somatostatin, and PP, respectively. Insulin production is limited to beta-cells. The homeodomain transcription factor pancreatic duodenal homeobox-1 (PDX1) and insulin were used as markers in the characterization of the human fetal pancreatic clones.

According to the original Edmonton Protocol (8) a minimum of 4000 islet equivalents per kilogram of the recipient's body weight in a packed-tissue volume of less than 10 ml are necessary for transplantation. The same group reported in a later study (8) that 17 patients all became insulin independent after a minimum of 9000 islets/kg was transplanted. Pancreatic cells according to the present invention are able to form islet equivalents (referred to herein as aggregates) when cultured using untreated Petri dishes. Three million pancreatic cells in 8 ml culture medium/90 mm Petri dish can produce 35000 aggregates. This procedure can be scaled up to produce a sufficient quantity of aggregates to be used in human transplantation, following procedures developed for diabetic patients known in the art such as those described by Shapiro and colleagues (2).

As used herein, the term “immortal” refers to a cell with the ability to undergo extended proliferation. The pancreatic cells of the current invention are immortal when grown in the presence of 4-OHT. A culture of cells in vitro, expanded from a single cell or a colony of cells, is referred to as a “cell line”, as will be appreciated by one skilled in the art. This is in contrast to primary cells, which can only divide a limited number of times, normally less than 10-20 divisions, before senescence is reached and the cell eventually dies. As used herein, the term conditionally immortal refers to a cell that is dividing and immature under certain, specific, growth conditions but which is a fully mature and non-dividing cell under other conditions. According to the current invention, the environmental condition responsible for immortalizing the cells is the presence of the estrogen receptor ligand.

The feature of the pancreatic cells that allows them to be continually immortal, and therefore be immortalized in the presence of 4-OHT, is the presence of a myc/estrogen receptor fusion protein. Without wishing to be bound by theory, it appears that the 4-OHT activates the estrogen-receptor. Activation of the estrogen-receptor allows the oncoprotein myc to dimerise and be transported into the nucleus where it acts as a transcription factor, initiating expression of genes allowing proliferation and genetic stabilization to occur (see for example Pollock et al., 2006; reference 9). Without 4-OHT, the fusion protein comprising the myc oncoprotein is still expressed but it remains in the cytoplasm and no further proliferation occurs. The ligand can therefore be added to the media to make the pancreatic cells proliferate (immortally), and can be withdrawn allowing the cells to behave like normal non-proliferating pancreatic cells and differentiate into functional islet cells.

The pancreatic cells of the invention are conditionally immortal due to the expression of a myc/estrogen-receptor fusion protein. As used herein, the term “fusion protein” refers to a recombinant protein that comprises two protein or peptide sequences that are naturally expressed separately, expressed as a single polypeptide chain.

The fusion protein may comprise any myc protein and any estrogen-receptor, or any fragments of these proteins that maintain the ability to be activated by the oestrogen receptor 4-OHT and activate transcription leading to cell proliferation, respectively. Preferably, the myc protein is c-myc. Preferably the estrogen-receptor has a mutation that prevents high affinity binding to 17 beta-estradiol, without affecting the high-affinity binding to 4-OHT. This mutation may be a deletion, substitution or addition of one or a number of amino acid residues. In a preferred embodiment, the fusion protein consists of a human c-myc gene fused to the mouse estrogen receptor. More preferably, the fusion protein comprises the amino acid sequence identified herein as SEQ ID No. 2. As stated previously, any homologue or functional fragment of SEQ ID No. 2 is within the scope of the invention.

As used herein, the term “homologue” refers to the similarity or identity between two or more biological polymers, including DNA, RNA and protein sequences. The concept of sequence identity is well known in the art, and refers to the level of identity between two sequences. Equally well known is the concept of similarity, wherein conservative differences between two sequences, which do not have a large effect on structure or function, are included when considering the likeness between two sequences. For example, a glutamic acid may be substituted for an aspartic acid without a large effect on the protein structure or function; these residues are “similar”. In contrast, an aspartic acid residue shows no similarity to a phenylalanine residue. Homologues included within the scope of the invention must have a high similarity to the mouse estrogen receptor and human c-myc sequences identified herein. Identity and similarity may be calculated using any well-known algorithm, for example Needleman-Wunsch, Smith-Waterman, BLAST or FASTA. Homology may be determined at the nucleic acid or amino acid level. Preferably, homologues within the scope of the invention have at least 50%, more preferably at least 60%, even more preferably greater than 70% and most preferably greater than 80%, for example 90%, 95%, 96%, 97%, 98% or 99% homology at the amino acid or nucleic acid level as calculated using the BLAST programme (Atschul et al, J. Molec. Biol., 1990; 215:403-410) under default conditions.

The terms “variant”, “homologue”, “derivative” or “fragment” as used herein include any substitution, variation, modification, replacement, deletion or addition of one (or more) amino acid from or to a sequence. The variant may have a deletion, insertion or substitution variation that produces a silent change and a functionally equivalent polypeptide. Deliberate amino acid substitutions may be made on the basis of similar physio-chemical properties such as size, charge and hydrophobicity. Conservative substitutions may be made, for example according to the table below. Amino acids in the same block in the second column and preferably in the same line in the third column may be substituted for each other

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The pancreatic cells of the invention express the c-myc/estrogen receptor fusion protein. The polynucleotide molecule encoding the fusion protein, referred to herein as the “fusion polynucleotide”, may be present in the pancreatic cells in any form, for example as a plasmid within the pancreatic cells or integrated into the host's genome.

It is preferred that the pancreatic cells are conditionally immortalized by incorporation of the fusion polynucleotide into the genome. Methods for the integration of heterologous polynucleotides into a host genome are well known in the art and any suitable method may be used. Preferably, retroviral infection is used to integrate the fusion polynucleotide into the genome. Retroviral vectors for the integration of genetic material into foreign genomes are well known in the art, and any may be used. Preferably, the vector is an amphotropic retrovirus, most preferably, the vector is pLNCX (BD Biosciences Clontech).

The vector is packaged together with the fusion polynucleotide in any suitable virus producing cells. The virus is preferably produced by Fly-CO42 cells originated from the TEFLY virus producer cell line.

The pLNCX vector comprises a LTR promoter, which drives a neomycin resistance gene. Neomycin (also known as geneticin and G418) is used in the media during selection so that only cells expressing the neomycin resistant gene survive. A titration of neomycin can be performed on non-infected target cells to establish the concentration required to eliminate uninfected cells. Any antibiotic resistance gene may be used in order to aid selection of infected cells, although antibiotic resistance is not essential.

Any promoter can be used to promote expression of the fusion polynucleotide. Preferably, this is a different promoter to that used to promote expression of any antibiotic resistance gene. Preferably, a CMV promoter drives expression of the fusion polynucleotide.

An example of a suitable fusion polynucleotide is c-mycERTAM, identified herein as SEQ ID No. 1, comprising a human c-myc gene fused to a mouse oestrogen receptor that is mutated to remove high affinity binding to 17 beta-estradiol without affecting the high affinity binding to the synthetic drug 4-OHT. Any mutation to the polynucleotide that causes this functional change in the protein is within the scope of the invention, including an addition, substitution or deletion. Preferably, the mutation is a point mutation. In the preferred embodiment, a point mutation is introduced to alter the wild-type glycine at amino acid position 681 to arginine. Homologues and functional fragments of c-mycERTAM are within the scope of the invention.

It will be apparent to one skilled in the art in order for the retroviral vector to integrate into the pancreatic cell genome; the pancreatic cells need to be proliferating. To maximise integration, the virus should be added when the pancreatic cells are proliferating at a high rate. To further increase the success rate for the infection, a facilitator like hexadimethrine bromide (also known as polybrene) may be added to the media during infection. This can be toxic to some cells at high concentrations, but it has been successfully used for infection of human fetal pancreatic cells (7)

An advantage of using 4-OHT as the proliferation signal in pancreatic cells, according to the invention, is that this synthetic chemical is not normally present in humans, allowing the pancreatic cells, according to the invention, to be used as a medicament. In particular, the pancreatic cells can be used in therapeutic cell lines, for use in transplantation therapy. It is recognised that transplantation of pancreatic cells is an option to treat diseases of the pancreas, where transplantation of healthy pancreatic cells into a diseased or damaged pancreas can replace cells damaged by disease. Cell transplantation is seen as a preferable alternative to organ transplantation. Any disease that impairs pancreas function may be treated by the transplant of pancreatic cell or cell aggregates according to the invention, including but not limited to diabetes, and cancer. Veterinary treatments involving the pancreatic cells are also within the scope of the invention.

It will be readily apparent to the skilled person that the cells of the invention may be transplanted using conventional cell and islet transplantation technologies. The cells may be introduced using any suitable technique. Conventional immunosuppressants may also be administered, as is done for regular transplantation treatments. The preparation of suitable compositions intended for therapeutic use will be apparent to the skilled person.

The cells, cell lines and cell aggregates of the invention are useful in to screening assays to evaluate the toxicology of potential drugs or to establish the effectiveness of a drug in a particular treatment. Suitable screening assays will be apparent to the skilled person. The assays may be used to evaluate changes to the function, morphology or genetic structure of the cells of the invention when brought into contact with a drug.

Typically, the method for evaluating the suitability of a compound for use as a drug, comprises the steps of:
(i) contacting a cell or cell aggregate according to the invention with the potential drug compound; and
(ii) measuring the response of the cell, or cell aggregate, to thereby determine the effect of the compound on the cell or cell aggregate body and evaluate the suitability of the compound for use as a drug.
   The intention may also be to identify compounds which act to stimulate the cells to produce insulin in response to glucose. Alternatively, the intention may be to identify compounds which stimulate the cells to mature upon cell transplantation. Alternatively, the intention may be to identify compounds which stimulate islet cells, to produce glucagons, somatostatin and/or pancreatic polypeptide.

The invention will now be described further in the following non-limiting examples.

### Example

All chemicals were purchased from Sigma unless otherwise mentioned.

### Tissue Culture

All cells were grown at 37° C. in 5% CO₂ incubators. The cells were fed on a regular basis with media change three times a week. The cells were passaged when at 70-95% confluency.

### Media

Human fetal pancreatic cells and clones were cultured in medium called Human Pancreas Medium (HPM) which consisted of the following alternatives:

(1) MegaCell™ Dulbecco's Modified Eagle's Medium Nutrient Mixture F-12 Ham, 1.5% fetal calf serum (FBS) (HyClone), and 2 mM L-glutamine. The medium was sterile filtered with 1 L filter units (Nalgene) and pre warmed to 37° C.; or

### (2) CMRL Connaught Medical Research Laboratories Media; or

### (3) UltraCulture™ (Cambrex);

Before use, the following growth factors (GF) were added to HPM: 20 ng/ml epidermal growth factor (EGF) and 50 nM Gastrin I. After infection the pancreas cells were cultured in the presence of a supplement: 100 nM 4-Hydroxytamoxifen (4OHT).

### Coating

Tissue culture treated plasticware or uncoated plasticware were used. In some cultures the coating was done with a solution of fibronectin, diluted in sterile bottled water to a final concentration of 100 μg/ml. The plastic ware was washed finally in HBSS before use. In other cultures, no extracellular matrix substrates were used.

### Pancreas

Human fetal pancreases were obtained following terminated pregnancies. Local NHS ethical committee approval had been obtained prior to the research being started.

### Dissociation

The human fetal pancreas was received on wet ice in RPMI media after shipment. The pancreas was washed in 4° C. calcium-free hanks balanced salt solution (HBSS) (Gibco). The pancreas was minced with scalpels. The minced fetal pancreas was digested with collagenase P (Boehringer Mannheim), incubated for 15 min at 37° C., agitated and triturated every 2 min.

The dissociated pancreas cells were counted and cell viability evaluated using a hemocytometer. In one example, the cells were plated in HPM and GFs on untreated Petri dishes plasticware that discourage cell attachment and left for 3-5 days to allow the formation of islet cell clusters (ICCs)/aggregate formation, selecting preferentially for islet cell progenitors. In another example, this selection was omitted and dissociated cell preparations were plated directly onto tissue culture ware.

### Harvesting of Virus

Virus producer cells from clone Fly-C042 were cultured to confluence in several T-175 flasks. Virus was harvested in HPM. When confluent, the flasks were washed 3× with PBS, and HPM was added to the virus-producing cells for 8 hours, 18 ml/T175 flask. The media was harvested and filtered through a 0.45 μm filter and aliquoted, 5 ml media/falcon tube, and snap frozen in liquid nitrogen and stored in −80° C.

### Infection

Pancreas cells or preselected pancreas cell aggregates were transferred on fibronectin coated or uncoated vessels. Pancreatic cells were infected with virus supernatant and 4-8 μg/ml hexadimethrine bromide. After 8-16 hours of infection, fresh HPM was added and the infection cycle was repeated the next day. The infection was done in 10 cm Petri dishes with cell confluency ranging from 40% to 70%. After infection 4OHT was added to the medium. After 2 days from completed infection the cells were passaged for expansion and/or selection.

### Selection

The infected pancreatic cells were passaged and plated at low density on 16 cm Petri dishes and selected with 100-300 μg/ml Geneticin for 2 weeks. After selection the cells positively selected had formed individual clones, these clones were collected and expanded to originate pancreatic cell lines.

### Passaging

Each time the cells were passaged or removed from a flask the flask was washed once with 37° C. HBSS. After that a 37° C. 1× trypsin-versene mix (Bio Whittaker) was added for 2-5 minutes and the cells detached from the plastic. An equal volume of media was added to the trypsin-versene mix to neutralise the trypsin and the cells were spun for 5 minutes at 500 g in a falcon tube. The supernatant was aspirated and the cells resuspended in 1 ml of media and a cell count was preformed with a hemocytometer. The cells were passaged into a new freshly fibronectin-coated or uncoated vessel.

### Freezing/Thawing

When freezing the cells the cell pellet was resuspended in 900 μl of media and 100 μl of cryosure-dimethyl sulfoxide (DMSO) (Quest biomedical) in a cryo-safe vial (Nunc). The cells were stored at −80° C. for 24 hours in a Mister Frosty cryopreservation freezing container (Nalgene), where the temperature decreased at approximately 5° C./minute. After 24 hours the cells were moved to liquid nitrogen for long-term storage. When thawed, the cells were removed from the liquid nitrogen and placed in a 37° C. water bath until completely thawed or directly plated in the vessels or mixed with 10 ml of 37° C. media and:spun for 500 g for 5 minutes. The supernatant was discarded and the pellet resuspended in fresh media by gentle pipetting and plated in a new vessel.

### Immunocytochemistry

For early assessment of the cells, immunocytochemistry was used. It was not possible to quantify what the expression level was but the benefit with immunocytochemistry was that few cells were needed and it was possible to see how many cells expressed a specific marker in a cell population. It was also possible to see if some cells had higher expression than others. This is of great value in the process of choosing cells and cell lines to go forward with and which cell lines to discard.

Antibodies used: guinea pig anti-insulin (Chemicon/Sigma) and rabbit anti-PDX1 antibody (Chemicon). All staining was performed in multi-well format and the following procedures were used. The media was aspirated and the cells were washed once with PBS and fixed in 4% paraformaldehyde in PBS for 15 minutes at room temperature (RT). The 4% paraformaldehyde was aspirated and the cells washed 3 times with PBS. The cells were permeabilized for 20 minutes in 0.1% triton x-100 in PBS. The cells were then washed once with PBS and blocked, 30 minutes, in 10% normal goat serum (NGS) (Vector) in PBS. Primary antibodies were added in 1% NGS in PBS for over night at room temperature. The wells were washed 3×5 minutes with PBS and the secondary antibody was added in PBS for 2 hour at room temperature. The secondary antibody excited at a wavelength of ˜488 nm and gave emission at ˜525 nm (green emission). After 2 hour incubation the wells were washed 3×5 minutes in PBS and Hoechst, a chemical that stains DNA was added for 2 minutes at room temperature, 1:25 000 in PBS. Hoechst excited at ˜350 nm and gave emission at ˜425 nm (blue emission). Finally the wells were washed 3×5 minutes in PBS and left in the last wash. All analysis was done with the fluorescence microscope Leica DMRA and the digital camera C4742-95 (Hamamatsu) and computer software-Hamamatsu image PRO.

### Total RNA Extraction

For the total RNA extraction the RNeasy mini kit (Qiagen 74104) was used. A cell pellet of up to 5×10⁶ cells was used per extraction. The cells were disrupted with 350 μl RLT buffer with 2 mercaptoethanol, 10 μl/ml, and the sample homogenized by trituration with Gilson pipette. Absolute alcohol (Joseph Mills Ltd) was diluted to 70% and 350 μl added to the sample and mixed by pipetting. The sample was transferred to an RNeasy mini column and spun at 8000 g for 15 s. 350 μl RW1 wash buffer was added and spun at 8000 g for 15 s. 10 μl DNAse 1 RNAse-free (Qiagen 79254) in 70 μl RDD buffer (Qiagen) was added to the RNeasy mini column membrane for 15 minutes. 350 μl RW1 wash buffer was added and spun at 8000 g for 15 s, 500 μl RPE buffer was added twice and the sample spun for 15 seconds and 2 minutes at 8000 g each time. Finally 50 μl RNAse-free water was added to the column and spun at 8000 g for 1 minute. The 50 μl water containing the RNA was collected in a Rnase-free tube and purity and quantity was checked with the spectrophotometer GeneQuant pro (Amersham pharmaceutical biotech AB). The sample was stored at −80° C.

Making cDNA

To make cDNA, extracted total RNA was thawed on ice and 50 ng was mixed with the following in DNase/RNase free tubes on ice: RNasin RNase inhibitor 10000 u, (Promega N2115), 0.25 μl, 10M random primers (Invitrogen 48190-011) or 10 μM oligodT, 2 μl, and buffer RT 10×, 2 μl, dNTP-mix 5 mM each, 2 μl, Sensiscript RT, 1 μl, and RNase-free water (all from Quiagen kit 205213) were mixed to a final volume of 20 μl. The tubes were placed in a 37° C. water bath for 60 minutes, transferred to a 93° C. heat block for 5 minutes and rapidly cooled on ice. The cDNA was stored in a 20° C. freezer.

### RT-PCR

For the PCR of the c-myc-ER and insulin, the titanium taq PCR kit (Clontech laboratories) was used, see Table 2 for the primers used. The PCR reaction conditions were standard. The PCR machine used was GeneAmp PCR system 2700. For analysis, 15 μl of the PCR product was mixed with 5 μl 6× loading dye solution (MBI R0611) and loaded to a gel. A 100 bp DNA ladder (MBI SM0241), 6 μl, containing 3 μg DNA was also loaded. The gel was a 2% agarose gel consisting of: TAE buffer (Invitrogen 15558-034), agarose 2%, ethidium bromide, 267 μg/ml. The gel was run with an electrophoresis power supply E835 (Consort) in a tray HU13 (Jencons) and finally scanned with Fluor-S multimager (Biorad) and analysed with the software Quantity One (Biorad). For quantitative PCR, total RNA is extracted from cell pellets using Trizol (Invitrogen) and RNeasy (Qiagen) spin-columns, prior to reverse-transcription with Superscript II (Invitrogen) to synthesize the first strand of cDNA, which can be used as a template in quantitative PCR. Each gene transcript is detected with a sequence-specific primer-probe combination which accumulates a fluorescent signal during each PCR cycle, detected using a Lightcycler 480 instrument (Roche); the transcription of each gene is normalized against loading-level by assessing a number of house-keeping genes, and expressed as a proportion of a calibrator sample.

TABLE 2
Primer pairs used for amplification of the c-myc-ER construct and insulin

| | | |
|---|---|---|
| | | Product |
| Gene | Sequence | size (bp) |

RT-PCR

| | | |
|---|---|---|
| c-myc 1235S/ | CTCCTGGCAAAAGGTCAGAG (SEQ ID NO. 3) | 590 |
| M-ER 1131AS | AAGGACAAGGCAGGGCTATT (SEQ ID NO. 4) | |
| c-myc 1585S/ | AAAGGCCCCCAAGGTAGTTA (SEQ ID NO. 5) | 240 |
| M-ER 1131AS | AAGGACAAGGCAGGGCTATT (SEQ ID NO. 6) | |
| Insulin S | CCTGTGGATGCGCCTCCTGC (SEQ ID NO. 7) | 401 |
| Insulin AS | GCTGGTTCAAGGGCTTTATTCCATCTC (SEQ ID NO. 8) | |

Quantitative PCR

| | |
|---|---|
| c-mycERTAM | Forward primer: agaggagcccagccagac (SEQ ID NO. 9) |
| | Reverse primer: tgtaaggaatgtgctgaagtgg (SEQ ID NO. 10) |
| NKX 6.1 | Forward primer: cgttggggatgacagagagt (SEQ ID NO. 11) |
| | Reverse primer: cgagtcctgcttcttcttgg (SEQ ID NO. 12) |
| ISL1 | Forward primer: aaggacaagaagcgaagcat (SEQ ID NO. 13) |
| | Reverse primer: ttcctgtcatcccctggata (SEQ ID NO. 14) |
| PDX1 | Forward primer: aagctcacgcgtggaaag (SEQ ID NO. 15) |
| | Reverse primer: gccgtgagatgtacttgttgaa (SEQ ID NO. 16) |
| Insulin | Forward primer: aggcttcttctacacacccaag (SEQ ID NO. 17) |
| | Reverse primer: cacaatgccacgcttctg (SEQ ID NO. 18) |
| | Or |
| | Forward primer: agaagaggccatcaagcaga (SEQ ID NO. 19) |
| | Reverse primer: caggtgttggttcacaaagg (SEQ ID NO. 20) |
| Nkx2.2 | Forward primer: cgagggccttcagtactcc (SEQ ID NO. 21) |
| | Reverse primer: ggggacttggagcttgagt (SEQ ID NO. 22) |
| Glucagon | Forward primer: gtacaaggcagctggcaac (SEQ ID NO. 23) |
| | Reverse primer: tgggaagctgagaatgatctg (SEQ ID NO. 24) |
| | Or |
| | Forward Primer: tctgttctacagcacactaccaga (SEQ ID NO. 25) |
| | Reverse Primer: agctgccttgtaccagcatt (SEQ ID NO. 26) |
| hTERT | Forward Primer: ggggtcactcaggacagc (SEQ ID NO. 27) |
| | Reverse Primer: tcttgaagtctgagggcagtg (SEQ ID NO. 28) |

### Aggregate Formation

Methods for generating aggregates were adapted from Gershengorn (10, 11). Pancreatic cells were expanded and plated on tissue culture-treated or untreated plasticware, with a minimum of 8×10⁵ cells per vessel in one of the following conditions as required: (a) 8 ml of human pancreas medium (HPM) plus growth factors including one or more of the following: Gastrin I (50 nM) and EGF (20 ng/ml); (b) 8 ml low glucose DMEM plus 1.5% HSA and growth factors; (c) 8 ml CMRL plus insulin (10 mg/l), transferrin (5.5 mg/l, selenium (6.7 μg/1) plus 1% HSA; (d) grown to confluent monolayer in treated tissue culture vessel in HPM, exposed 1-4 min to trypsin, followed by the addition of 8 ml CMRL plus insulin (10 mg/l), transferrin (5.5 mg/l, selenium (6.7 μg/l) plus 1% HSA;

The cells were incubated for a minimum of 24 hr, in a tissue culture incubator, 37° C. 5% CO₂, to allow aggregate formation.

### Glucose Shift

After a minimum of 24 hrs the aggregates were collected and transferred to 15 ml tubes. Centrifuged for 5×1500 rpm to remove supernatant and the pellets were washed using 10 ml of low glucose medium. The washed pellet were resuspended in 200 μl of DMEM low glucose, plus 2 mg/ml HSA and 10 mM hepes and incubated at 37° C. for 2 hrs. After 2 hrs the aggregates were collected by centrifugation (5×1500 rpm) and resuspend with 200 μl of DMEM high glucose, plus 2 mg/ml HSA, 10 mM hepes, and 10 mM theophylline and incubated at 37° C. for 2 hrs. After incubation the aggregates were collected by centrifugation (5×1500 rpm) and stored at −80° C.

### Human Insulin Detection by ELISA

20 μl of the pancreatic cell lysates and of control cells were used for human insulin ELISA kit (Linco cat.# EZHI-20K).

Human Proinsulin detection by ELISA

20 μl of the pancreatic cell lysates and of control cells were used for human total proinsulin ELISA kit (Linco cat.#EZHI-15K).

### Results

### Dissociation, Infection and Clone Selection

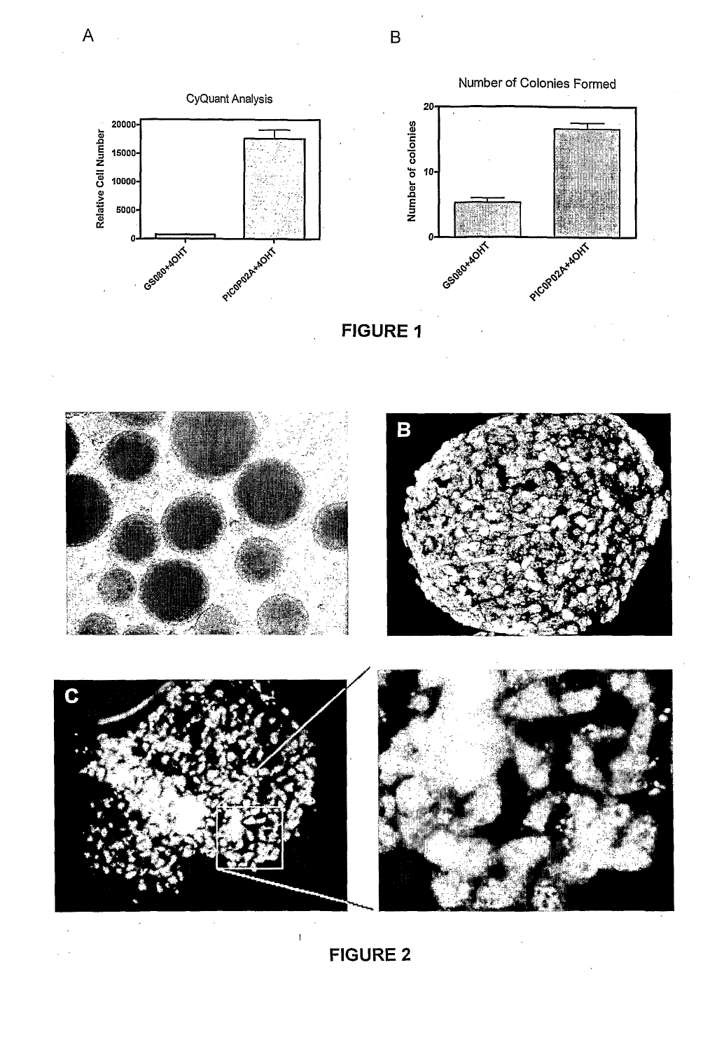

Human fetal tissue was received at 11-19 weeks gestation and dissociated. Following infection by c-mycERTAM, there was significantly enhanced cloning efficiency in c-mycERTAM infected cell populations than in uninfected control cells from the same tissue (see FIG. 1). Following 2 weeks selection, individual clones were picked and expanded. The clones that showed promising preliminary characteristics were subjected to further characterization. Two clones have been karyotyped at passage 20 and shown to be normal diploid cells.

### Immunocytochemistry

In cells containing the cmyERTam construct, addition of 4-OHT promotes proliferation, while removal of 4-OHT promotes differentiation of the cell to display the mature phenotype. A mature phenotype was observed on aggregated cells that underwent a glucose shift step. Certain clones showed positive staining for insulin and PDX1 and formed well-defined aggregates (see FIG. 2).

PCR for c-mycERTAM, Insulin and Pancreas-Specific Markers

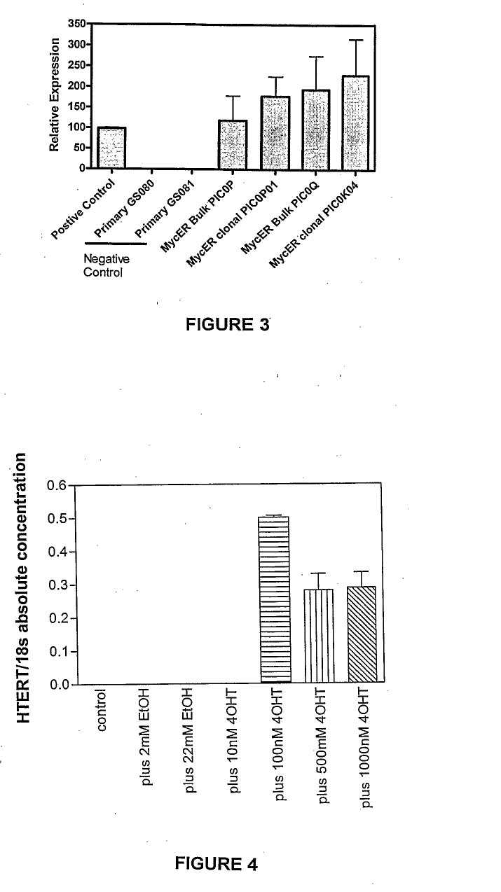

Quantitative PCR for c-mycERTAM and a Nuclease Protection Assay confirmed that the construct had integrated in all clones tested (FIG. 3). c-mycERTAM was shown to be functional by a c-myc gene target (Telomerase Reverse Transcriptase) induction by 4-OHT (FIG. 4).

PCR performed using the primers for insulin confirmed expression of insulin in aggregated clones exposed to glucose shift. Additional pancreatic islet markers as shown in Table 2 were also found to be positive.

### Insulin and Proinsulin Expression Detected by ELISA

Proinsulin ELISA detection performed on cell lysates from aggregated PIC0K04 cells, exposed to glucose shift, confirmed proinsulin expression.

Insulin ELISA detection performed on cell lysates from aggregated PIC0K04, PIC0K23 and PIC0K24 cells, exposed to glucose shift, confirmed insulin expression.

For the avoidance of doubt, all references referred to herein are hereby incorporated by reference.

### REFERENCES

(1) The Diabetes Control and Complication Trial Research Group: The effect of intensive treatment of diabetes on the development and progression of long-term complications in insulin-dependent diabetes mellitus. N Engl J Med 329:977-986, 1993
(2) Shapiro, A. M. J. et al. (2000) Islet transplantation in seven patients with Type 1 diabetes mellitus using a glucocorticoid-free immunosuppressive regime. N. Engl. J. Med. 343, 230-238
(3) Beattie G M, Otonkoski T, Lopez A D, Hayek A. 1997 Functional b-cell mass after to transplantation of human fetal pancreatic cells: differentiation or proliferation Diabetes. 46:244-248.
(4) Orci L, Unger R H 1975, Functional subdivision of islets of Langerhans and possible role of D cells. Lancet 2:1243-1244
(5) Orci L 1982, Macro- and micro-domains in the endocrine pancreas. Diabetes 31:538-565
(6) Otonkoski, T, Beattie G M, Mally M I, Ricordi C, Hayek A. 1993 Nicotinamide is a potent inducer of endocrine differentiation in cultured human fetal pancreatic cells. J Clin Invest. 92:1459-1466
(7) Leibowitz G, Bettie G M, Kafri T, Cirulli V, Lopez A D, Hayek A and Levine F. 1999 Gene transfer to human pancreatic endocrine cell using viral vectors. Diabetes. 48:745-753
(8) Edmond A. Ryan, Jonathan R. T. Lakey, Breay W. Paty¹, Sharleen Imes, Gregory S. Korbutt, Norman M. Kneteman, David Bigam², Ray V. Rajotte, and A. M. James Shapiro Successful Islet. 2002 Transplantation Continued Insulin Reserve Provides Long-Term Glycemic Control Diabetes 51:2148-2157.
(9) (10) Gershengorn et al, Science 306:2261-2264.
(11) Hardikar et al, PNAS (USA) 100:7117-7122.

1. A mammalian pancreatic cell comprising, as a single polypeptide, a fusion protein comprising an oncoprotein of the myc family and an oestrogen receptor, or a functional fragment thereof.

2. The cell according to claim 1, wherein the oncoprotein is c-myc.

3. The cell according to claim 1, in culture media.

4. The cell, according to claim 1, which is a human cell.

5. The cell according to claim 1, comprising a polynucleotide encoding the fusion protein.

6. The cell according to claim 5, wherein the polynucleotide is integrated into the genome.

7. The cell according to claim 1, wherein the fusion protein comprises a mouse oestrogen receptor and a human c-myc protein.

8. The cell according to claim 1, wherein the oestrogen receptor 5 contains a mutation that prevents high affinity binding to 17β-oestradiol.

9. The cell according to claim 1, comprising the polynucleotide sequence identified herein as SEQ ID No. 1.

10. The cell according to claim 1, that expresses markers characteristic of a pancreatic cell in vivo.

11. The cell according to claim 10, wherein the markers include a combination of the following IPF1, insulin, NKX6.1, PDX, ISL1, NKX2.2, a glucagon and pancreatic polypeptide.

12. The cell according to claim 1, wherein the cell is conditionally immortalised when contacted with a ligand of the oestrogen receptor.

13. The cell according to claim 12 wherein the ligand is 4-hydroxytamoxifen.

14. A method for forming a cell aggregate body wherein said method comprises the use of a mammalian pancreatic cell comprising, as a single polypeptide, a fusion protein comprising an oncoprotein of the myc family and an oestrogen receptor, or a functional fragment thereof.

15. The method according to claim 14, wherein the cell aggregate body is formed on an uncoated surface.

16. A method of conditionally immortalising a cell, comprising the steps of:
(i) expressing in the cell a fusion protein comprising an oncoprotein of the myc family and an oestrogen receptor, or a functional fragment thereof expressed as a single polypeptide chain; and
(ii) contacting the cell formed by step (i) with a ligand of the oestrogen receptor, thereby conditionally immortalising the cell.

17. A method of evaluating the suitability of a compound for use as a drug, in vitro, comprising the steps of:
(i) contacting a cell according to claim 1, or a cell aggregate body formed with the cell, with the potential drug compound; and
(ii) measuring the response of the cell, or cell aggregate body, to thereby determine the effect of the compound on the cell or cell aggregate body and evaluate the suitability of the compound for use as a drug.

18. The method according to claim 17, wherein the compound stimulates the cell to produce insulin in response to glucose.

19. The method according to claim 17, wherein the compounds stimulates the cells to mature upon transplantation.

20. The method according to claim 17, wherein the compound stimulates islet cells.

21. The method according to claim 17, wherein the compound is evaluated for its toxicity to pancreatic cells.

22. (canceled)

23. Use of a mammalian pancreatic cell, or a cell aggregate formed with the cell, wherein the cell comprises, as a single polypeptide, a fusion protein comprising an oncoprotein of the myc family and an oestrogen receptor, or a functional fragment thereof, for the manufacture of a medicament for treating a disorder of the pancreas.

24-25. (canceled)

26. A method for treating a disorder of the pancreas wherein said method comprises using either
a) a mammalian pancreatic cell comprising, as a single polypeptide, a fusion protein comprising an oncoprotein of the myc family and an oestrogen receptor, or a functional fragment thereof, or
b) a cell aggregate body formed with the cell of part a).

27. The method, according to claim 26, wherein said disorder is diabetes.
